(19) United States
(12) Patent Application Publication
(10) Pub. No.: US 2007/0009909 A1
(43) Pub. Date: Jan. 11, 2007

(54) Sorting of carbon nanotubes through arrays

(76) Inventors: Herman A. Lopez, Sunnyvale, CA (US); Yuegang Zhang, Cupertino, CA (US)

Correspondence Address: BLAKELY SOKOLOFF TAYLOR & ZAFMAN, 12400 WILSHIRE BOULEVARD, SEVENTH FLOOR, LOS ANGELES, CA, 90025-1030, US

(21) Appl. No.: 11/173,761

(22) Filed: Jun. 30, 2005

(51) Int. Cl. C12Q 1/68

(52) U.S. Cl. 435/6; 977/924

(57) ABSTRACT

A method is described that involves sorting CNT hybrid structures of differing sizes by passing the CNT hybrid structures through an arrangement of fixed structures. The sorting involves a diffusion component in which shorter CNT hybrid structures are scattered through the arrangement of fixed structures to a greater degree than at which longer CNT hybrid structures are scattered through the arrangement of fixed structures.

### FIELD OF ART

The field of art relates generally to Carbon nanotubes (CNTs); more specifically, techniques for sorting CNTs of different electronic types.

### BACKGROUND

A Carbon nanotube (CNT) can be viewed as a sheet of Carbon that has been rolled into the shape of a tube. CNTs having certain properties (e.g., a “metallic” CNT having electronic properties akin to a metal) may be appropriate for certain applications while CNTs having certain other properties (e.g., a “semiconducting” CNT having electronic properties akin to a semiconductor) may be appropriate for certain other applications. CNT properties tend to be a function of the CNT's “chirality” and diameter. The chirality of a CNT characterizes its arrangement of carbon atoms (e.g., arm chair, zigzag, helical/chiral). The diameter of a CNT is the span across a cross section of the tube.

Because the properties of a CNT can be a function of the CNT's chirality and diameter, the suitably of a particular CNT for a particular application is apt to depend on the chirality and diameter of the CNT. Unfortunately, current CNT manufacturing processes are only capable of manufacturing batches of CNTs whose tube diameters, lengths and chiralities are widely varied. The problem therefore arises of not being able to collect CNTs (e.g., for a particular application) of only a particular size range (e.g., length and/or diameter range(s)) and/or electronic property (e.g., metallic or semiconducting) from a batch of manufactured CNTs having widely varied sizes of both metallic and semiconducting CNTs.

CNTs are also known to have poor solubility. Here, owing to van der Waals forces (it is believed), individual CNTs tend to “bundle together” into groups. Thus, when a batch of manufactured CNTs are made to flow in a fluidic stream (such as an aqueous solution), bundled groups of CNTs are observed drifting/flowing through the liquid together.

Success at improving the solubility of CNTs has been reported. For example, Zheng et al. (“DNA-Assisted Dispersion and Separation of Carbon Nanotubes”, Nature Materials 2, pgs. 338-342, 2003) has published a process by which single stranded DNA (ss-DNA) is used to “break-down” a CNT bundle into individual CNTs wrapped in a helical structure of DNA. Here, a CNT that has bonded in some fashion with DNA so as to form a combined structure of DNA and the CNT is referred to as “DNA/CNT hybrid structure”. A DNA/CNT hybrid structure of DNA and a metallic CNT may be referred to as “DNA/metallic CNT hybrid structure”. A DNA/CNT hybrid structure of DNA and a semi-conducting CNT may be referred to as “DNA/semi-conducting CNT hybrid structure”. A “CNT hybrid structure” can be viewed as a CNT that is attached to another substance.

According to the technique taught by Zheng et al., an aqueous solution containing bundles of CNTs is subjected to the presence of ss-DNA. Because the binding energy associated with the coupling of ss-DNA to a CNT is comparable to the binding energy associated with the coupling of CNTs to one other, the application of sonic energy to the solution can create dynamic situations in which an individual CNT that is bundled with one or more other CNTs will reach a lower energy state if the CNT binds with ss-DNA molecules instead of the CNTs associated with its bundle. Because physical systems tend to fall to lower energy states, this prompts the formation of an individual (i.e., non bundled) CNT helically wrapped in ss-DNA. That is, the CNT essentially leaves its bundle in favor of being helically wrapped by ss-DNA.

According to follow-up work reported by Zheng et al. in “Structure-Based Carbon Nanotube Sorting by Sequence Dependent DNA Assembly”, Science 28 Nov. 2003; 302: 1545-1548, a particular sequence of ss-DNA can be made to self assemble into a helical structure that wraps around the surface of an individual CNT. Individual CNTs wrapped by ss-DNA can then be sorted according to their electrical characteristics through anion exchange chromatography. In this manner, individual CNTs having specific “sought-for” electrical characteristics can be collected.

### FIGURES

The present invention is illustrated by way of example and not limitation in the figures of the accompanying drawings, in which like references indicate similar elements and in which:

FIG. 1a shows a flow of CNTs being injected into an array;

FIG. 1b shows the flow of FIG. 1a emerging on the other side of the array;

FIG. 2 shows an emergence profile for various types of CNTs that flow through an array;

FIGS. 3a through 3d show various types of arrays;

FIGS. 4a and 4b shows a system for sorting CNTs that contains an array.

### DETAILED DESCRIPTION

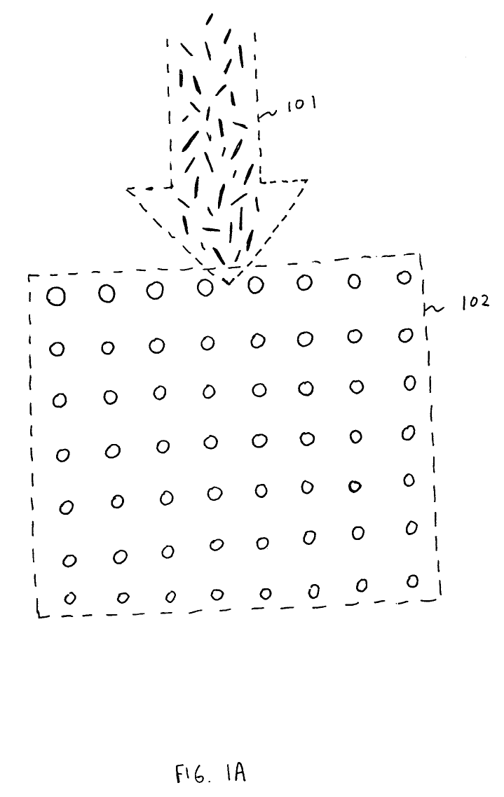

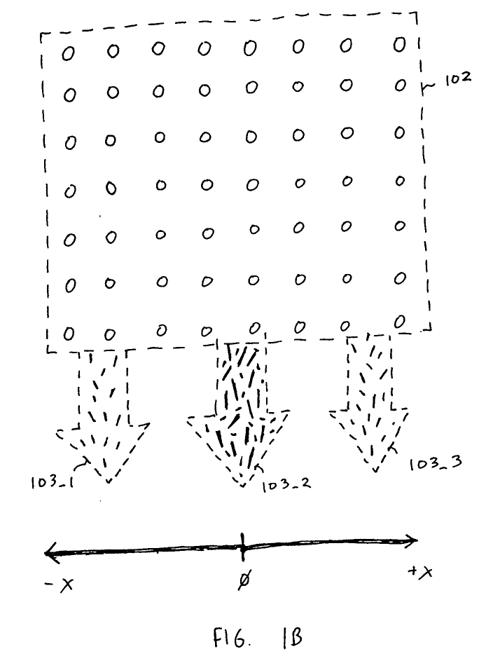

A technique for sorting a batch of metallic and semiconducting CNTs of differing lengths and diameters is to flow them through an arrangement of fixed structures such as an array of posts or studs. Owing to the dynamics by which the CNTs flow through the array as described in more detail below, CNTs of certain size and electronic property type will emerge from the array at a certain time and/or location. FIGS. 1a and 1b show a high level perspective.

According to FIG. 1a, a flow 101 of metallic and semiconducting CNTs of various lengths and diameters are observed flowing into the middle of an “entrance” edge of an array 102. FIG. 1b shows the flow of CNTs at a moment of time after that depicted in FIG. 1a. Importantly, as observed in FIG. 1b, the smaller CNTs are observed emerging 103_1, 103_3 from an “emission” edge of the array 102 farther out from the middle of the array 102 than a mixture of larger and smaller CNTs 103_2. Therefore the act of flowing various CNTs through an array can be used as a basis for separating the CNTs into categories of likeness (e.g., by gathering smaller CNTs at the outer edges of the “emission” edge 102).

Two basic separation principles that may simultaneously affect the flow of CNTs through the array include: 1) smaller CNTs (principally based on CNT length rather than CNT diameter) will diffuse farther out along the edge of the array than larger CNTs (as alluded to above with respect to FIGS. 1a and 1b); and, 2) if the array's posts are designed to promote some type of electric field based interaction with the DNA/CNT hybrid structures, semiconducting hybrid structures are more apt to be attracted to the array's posts than metallic hybrid structures resulting in hybrid structures with metallic CNTs emerging from the array farther out along the array edge and earlier in time than hybrid structures with semiconducting CNTs of comparable length and diameter.

The first principle above stems from basic principles of diffusion. Here, larger CNTs (principally, longer CNTs) will diffuse less in comparison to smaller CNTs and upon collision with the posts they will be confined to the vicinity of the center of the array. Here, both smaller and larger CNTs will collide with the array, but smaller CNTs with collide with posts further from the center of the input stream as a result of their larger diffusion; which, in turn, effectively corresponds to the outward surge of the larger CNTs, as a whole, being suppressed by the array posts to a greater extent than the collective outward surge of the smaller CNTs. If the rate of the total flow of CNTs through the array is less forcibly induced (e.g., with a “slow” fluidic flow through the array), it is also possible that smaller CNTs will diffuse out of the array earlier in time than the larger CNTs.

The second principle described above is a consequence of the fact that semiconducting CNTs do not promote the formation of “image” charges on the CNT surface as prevalently as metallic CNTs do. Here, as is well known in the art, metals form image surface charges of a first polarity in the presence of charges of opposite polarity. For example, the presence of a positive charge proximate to a metal will cause negative image charges to appear at the metal's surface. The ability to form such image charges is directly related to the very high mobility of electrons within metals. Because semiconducting materials do not possess electron mobilities as high as metals, semiconducting materials do not form image charges as strongly as a metallic materials.

As such, metallic CNTs are apt to have a greater propensity for producing image charges on their respective surfaces than semi-conducting CNTs. In this regard, an additional sorting mechanism may occur if the CNTs are individually wrapped in DNA as described in the background section of the present application. Specifically, because strands of DNA are negatively charged, DNA/metallic CNT hybrid structures are apt to be neutrally charged (because the underlying metallic CNT will have induced positive image charges that effectively cancel the DNA's negative charge), while, DNA/semiconducting CNT hybrid structures are apt to be negatively charged (because the underlying semiconducting CNT will not have induced sufficient positive image charges to effectively cancel the DNA's negative charge).

As a consequence, if the array's posts are given an electrical charge, DNA/metallic CNT hybrid structures are apt to be unaffected (i.e., there will be little if any attraction/repulsion between the hybrid structure and the array post), while, DNA/semiconducting CNT hybrid structures are apt to exhibit some kind of attraction to or repulsion from an array post depending on the array post's charge (specifically, attraction if the post is charged positively and repulsion if the post is charged negatively). In the case of attraction, the DNA/semiconducting CNT hybrid structures will: 1) progress slower through the array than the DNA/metallic CNT hybrid structures; and, 2) will have their outward surge thwarted to a greater degree than the DNA/metallic CNT hybrid structures. As a consequence, hybrid structures with semiconducting CNTs will emerge from the array later in time and closer to the entrance position of the input flow than hybrid structures with metallic CNTs.

In the case of repulsion (i.e., where the array posts are negatively charged), the opposite separation dynamics are apt to occur. Specifically, the DNA/semiconducting CNT hybrid structures will: 1) progress faster through the array than the DNA/metallic CNT hybrid structures; and, 2) will have their outward surge promoted to a greater degree than the DNA/metallic CNT hybrid structures. As a consequence, hybrid structures with semiconducting CNTs will emerge from the array sooner in time and farther from the entrance position of the input flow than hybrid structures with metallic CNTs.

Moreover, as reported in the work by Zheng, because DNA wrapping geometries change as a function of CNT diameter, the larger the diameter of a CNT within a hybrid structure, the more the CNT will behave like an electric dipole. A CNT in the form of an electric dipole can be viewed as, owing to the presence of an electric field within the CNT, one tip of the CNT being positively charged and the other tip of the CNT being negatively charged.

Here, if the array's posts are positively charged, an electric dipole CNT will position itself so that its negative tip is oriented toward an array post. This effectively corresponds to a form of attraction between the dipole-like CNT and the array post, which, similar to the attractive force described just above, slows the rate at which the CNT can flow through the array and shorten the distance the CNT will emerge outward along the array edge. Because semiconducting CNTs are more apt to exhibit dipole-like behavior than metallic CNTs (because semiconducting CNTs are more dielectric-like than metallic CNTs and are therefore more capable of sustaining an internal electric field), similar separation dynamics may occur in which hybrid structures with semiconducting CNTs emerge later in time and closer to the array entrance position than hybrid structures with metallic CNTs (assuming the CNTs for both kinds of hybrid structures have similar lengths and diameters).

At a high level, the principles described above can be summarized as demonstrating that: 1) principles of diffusion will promote smaller Hybrid structure to emerge farther out along the array emission edge (and perhaps sooner than) larger Hybrid structure; and, 2) if the arrays are “treated” so as to promote some type of attraction or repulsion interaction with specific types of Hybrid structures, the Hybrid structures that exhibit the strongest attraction to the array posts are apt to emerge later in time and closer to the array entrance point than other Hybrid structures, or, contra wise, if the array posts are treated so as to promote repulsion interaction with specific types of Hybrid structures, the Hybrid structures that exhibit the strongest repulsion to the array posts are apt to emerge sooner in time and farther out along the array's emission edge than other Hybrid structures.

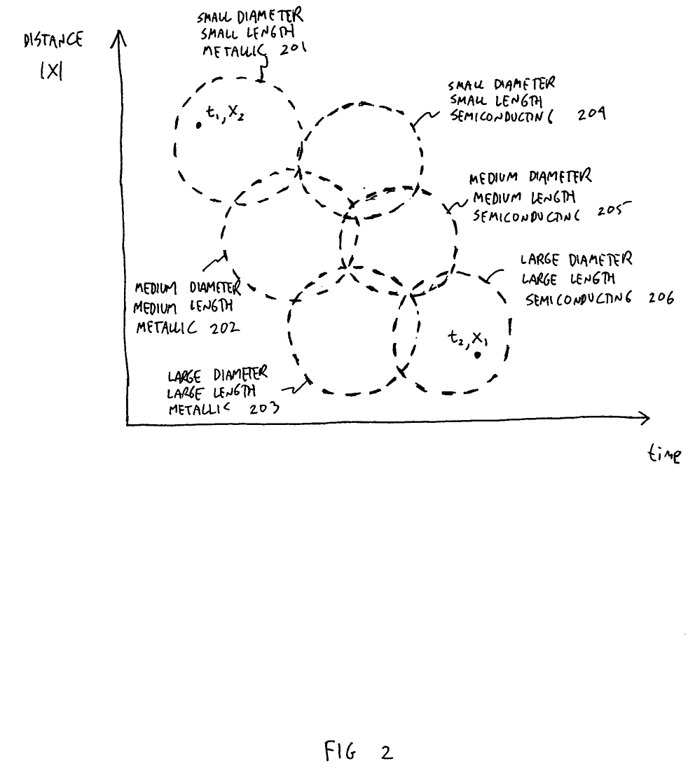

FIG. 2 further elaborates on these principles. Specifically, FIG. 2 plots array emergence position as a function of time for a flow of DNA/CNT hybrid structures through an array of positively charged posts where a slow flow rate exists so as to enhance the impact of pure diffusion on the sorting dynamics. Here, consistent with the principles discussed above, specific groups of hybrid structures 201 through 206 are observed as having specific, respective ranges of: 1) time of emission from the array (horizontal axis); and, 2) location along the emitting edge of the array (vertical axis where a position that is “higher up” on the vertical scale corresponds to a position that is farther out along the emitting edge of the array than the position on the entrance edge of the array where the flow of CNTs are introduced).

According to FIG. 2, longer, larger diameter, semiconducting CNT based hybrid structures 206 generally emerge later in time and closer to the entrance position of the array than the other hybrid structures; and, shorter, smaller diameter, metallic CNT based hybrid structures 201 emerge sooner in time and farther out along the array's emission edge than the other hybrid structures. Here, shorter, smaller diameter metallic CNTs could easily be collected by collecting hybrid structures at time t1 and position x2. Likewise, longer, larger diameter semiconducting CNTs could easily be collected by collecting hybrid structures at time t2 and position x1.

Notably, metallic CNT based hybrid structures 201 through 203 tend to emerge sooner than semiconducting CNT based hybrid structures 204, 205, 206 of comparable length and diameter. Amongst the metallic CNT based hybrid structures 201, 202, 203, the hybrid structures with shorter length and shorter diameter metallic CNTs 201 emerge farther out along the array and earlier in time than hybrid structures with medium length and medium diameter metallic CNTs 202; which, in turn, emerge farther out along the array and earlier in time than hybrid structures with longer length and longer diameter metallic CNTs 203.

Likewise, amongst semiconducting CNT based hybrid structures 204, 205, 206, the hybrid structures with shorter length and shorter diameter semiconducting CNTs 204 emerge farther out along the array and earlier in time than hybrid structures with medium length and medium diameter semiconducting CNTs 205; which, in turn, emerge farther out along the array and earlier in time than hybrid structures with longer length and longer diameter semiconducting CNTs 206.

In this regard it is important to recognize that although only electric field array interactions have been described above (e.g., by positively or negatively charging the array's posts), other types of attractive or repulsive interactions are possible. For example, the array posts may be treated so as to promote chemical reactions with specific types of Hybrid structures. A good example is a simple addition reaction. For instance, a carbonyl group is attached to the tube and the array is coated with alcohol or amine groups that can attack the carbonyl double bond.

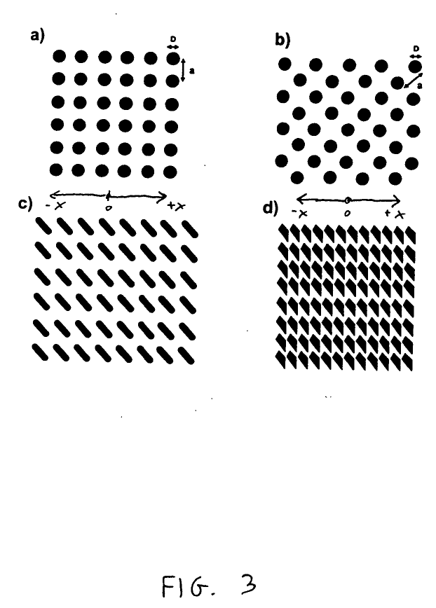

FIGS. 3a through 3d show different types of array designs that may be constructed to effect separation as described above. FIG. 3a shows an array design where, in both the x and y dimensions, all posts are aligned. FIG. 3b shows an array design where, in both the x and y dimensions, every other post is aligned. Both the array designs of FIGS. 3a and 3b are apt to spread out hybrid structures in both the +x and −x directions from an entrance point at the x=0 position.

The design of FIG. 3a, however, is apt to spread the hybrid structures out farther along the x axis than the design of FIG. 3b. Both the array designs of FIGS. 3c and 3d are apt to spread out hybrid structures in only the +x direction. The design of FIG. 3c, is apt to spread the hybrid structures out farther along the x axis than the design of FIG. 3d. Distances between array posts may vary from embodiment (e.g., ranging from microns to tens of nanometers (or less as manufacturing capabilities improve). Generally, the closer the array posts are to one another, the slower the hybrid structures will emerge from the array; and, the more spread out the hybrid structures will be when they emerge from the emitting edge of the array.

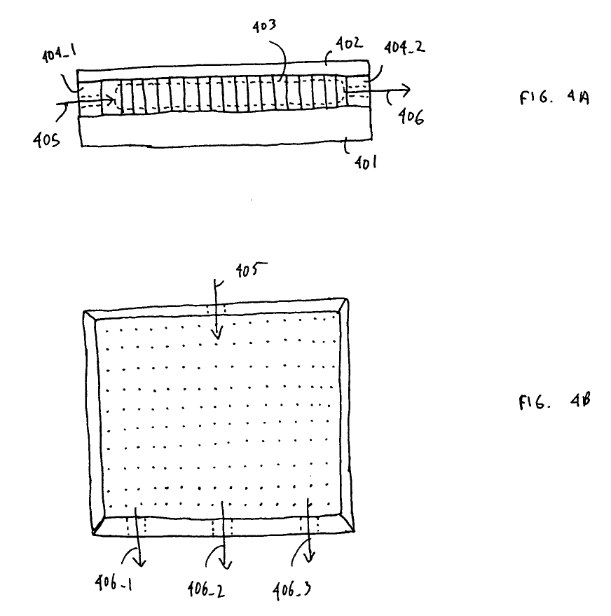

FIGS. 4a and 4b show a system for sorting CNTs as described above. According to the design of FIGS. 4a and 4b, post structures for the array 403 are formed with semiconductor integrated circuit manufacturing techniques (e.g., with lithographic patterned, stacked multilayer metal and/or dielectric features) upon a silicon substrate 401 and/or using standard PDMS (polydimethyl siloxane) microfluidic structures.

If electrical charge is to be applied to the posts, the posts should be made of metal that are interconnected with wiring at various locations. A lid 402 (e.g., formed with a ceramic and/or glass multi-layer structure) sits atop the array's posts. Sidewalls 404_1, 404_2 (e.g., again formed with lithographic patterned, stacked multiplayer metal and/or dielectric features) have embedded channels for introducing an input fluid flow 405 containing CNTs (e.g., wrapped into hybrid structures), and providing output fluid flows 406_1, 406_2, 406_3 at specific locations where specific types of CNTs are expected to emerge at specific times.

The process described above can also be multiplexed to increase separation efficiency. For example, if at the output of 406-1 of FIG. 4B another array is added (identically designed or having more optimized geometry for finer separation) a higher percentage of targeted CNTs will emerge from the second array.

In the foregoing specification, the invention has been described with reference to specific exemplary embodiments thereof. It will, however, be evident that various modifications and changes may be made thereto without departing from the broader spirit and scope of the invention as set forth in the appended claims. The specification and drawings are, accordingly, to be regarded in an illustrative rather than a restrictive sense.

1. A method, comprising:
sorting CNT hybrid structures of differing sizes by passing said CNT hybrid structures through an arrangement of fixed structures, said sorting comprising:
a diffusion component in which shorter CNT hybrid structures are scattered through said arrangement of fixed structures to a greater degree than at which longer CNT hybrid structures are scattered through said arrangement of fixed structures.

2. The method of claim 1 wherein said sorting further comprises
an attraction component in which CNT hybrid structures having a certain type of CNT are attracted to at least a portion of said fixed structures.

3. The method of claim 2 wherein said attraction component is effected through said portion of said fixed structures having a first charge and said CNT hybrid structures having said certain type of CNT have a second charge.

4. The method of claim 3 wherein said first charge is greater than said second charge.

5. The method of claim 4 wherein said first charge is positive and said second charge is negative.

6. The method of claim 2 wherein said attraction component is effected through a chemical reaction between said CNT hybrid structures having said certain type of CNT and said portion of said fixed structures.

7. The method of claim 6 wherein said chemical reaction causes said DNA/CNT hybrid structures having said certain type of CNT to be attracted to said portion of said fixed structures.

8. The method of claim 1 wherein said certain type of CNT is selected from the group consisting of:
metallic CNT; and,
semiconducting CNT.

9. A method, comprising:
sorting DNA/CNT hybrid structures of differing sizes by passing said DNA/CNT hybrid structures through an arrangement of fixed structures, said sorting comprising:
a diffusion component in which shorter DNA/CNT hybrid structures are scattered through said arrangement of fixed structures to a greater degree than at which longer DNA/CNT hybrid structures are scattered through said arrangement of fixed structures.

10. The method of claim 9 wherein said sorting further comprises:
an attraction component in which DNA/CNT hybrid structures having a certain type of CNT are attracted to at least a portion of said fixed structures.

11. The method of claim 10 wherein said attraction component is effected through said portion of said fixed structures having a first charge and said DNA/CNT hybrid structures having said certain type of CNT have a second charge.

12. The method of claim 11 wherein said first charge is greater than said second charge.

13. The method of claim 12 wherein said first charge is positive and said second charge is negative.

14. The method of claim 10 wherein said attraction component is effected through a chemical reaction between said DNA/CNT hybrid structures having said certain type of CNT and said portion of said fixed structures.

15. The method of claim 14 wherein said chemical reaction causes said DNA/CNT hybrid structures having said certain type of CNT to be attracted to said portion of said fixed structures.

16. The method of claim 9 wherein said certain type of CNT is selected from the group consisting of:
metallic CNT; and,
semiconducting CNT.

17. An apparatus for sorting CNT hybrid structures, comprising:
a fluid flow channel comprising a fluid flow input, multiple fluid flow outputs and an arrangement of fixed structures between said input and said outputs, said fixed structures situated upon a substrate, said arrangement of fixed structures spaced apart a distance within a range of:
microns to tens of nanometers.

18. The apparatus of claim 17 wherein said fixed structures comprise a multi-layer of stacked metal features.

19. The apparatus of claim 17 wherein a portion of said fixed structures are aligned along an axis.

20. The apparatus of claim 17 where said fixed structures are slanted to guide said CNT hybrid structures in a specific direction during said sorting.
